(19) United States
(12) Patent Application Publication
(10) Pub. No.: US 2010/0183703 A1
(43) Pub. Date: Jul. 22, 2010

(54) PHARMACEUTICAL OR COSMETIC COMPOSITION CONTAINING A DOUBLE STRANDED RNA OLIGONUCLEOTIDE AND ITS USE AS AN ACTIVE PHARMACEUTICAL INGREDIENT IN THE TREATMENT OF ANDROGEN RELATED DISEASES

(75) Inventors: Nestor Kerner, Buenos Airea (AR); Andrea Vanesa Dugour, Buenos Aires (AR); Maria Eugenia Balana, Buenos Aires (AR); Maria Carolina Alvarez-Roger, Buenos Aires (AR)

Correspondence Address: RATNERPRESTIA, P.O. BOX 980, VALLEY FORGE, PA, 19482, US

(73) Assignee: GEN-MED, S.A., Buenos Aires (AR)

(21) Appl. No.: 12/442,175

(22) PCT Filed: Sep. 19, 2007
(86) PCT No.: PCT/IB2007/053786

§ 371 Date: Jan. 12, 2010

(30) Foreign Application Priority Data
Sep. 21, 2006 (AR) P060104146

(51) Int. Cl. A61K 8/60; A61K 8/14; A61Q 7/02

(52) U.S. Cl. 424/450; 514/44 A

(57) ABSTRACT

This invention refers to new double stranded oligonucleotides having specific antiandrogenic activity and particularly to their use in the treatment of androgen-related diseases. Oligonucleotides may preferably be formulated with an acceptable pharmaceutical support in pharmaceutical or cosmetic formulations which are related to androgen metabolism or aimed at providing a beneficial effect on skin and/or hair.

This invention refers to new double stranded oligonucleotides having specific antiandrogenic activity and particularly to their use in the treatment of androgen-related diseases. Oligonucleotides may preferably be formulated with an acceptable pharmaceutical support in pharmaceutical or cosmetic formulations which are related to androgen metabolism or aimed at providing a beneficial effect on skin and/or hair.

### BACKGROUND OF THE INVENTION

By the end of the 60's, the manipulation of genetic material coming from different organisms set the foundations for what nowadays is called genetic engineering. By means of this technique, DNA sequences deriving from highly differentiated organisms (like viruses and bacteria) were joined into a single molecule.

In 1970 it was discovered that RNA retroviruses-viruses were capable of including foreign genetic sequences into their genome. These sequences probably came from the last infected cell and were then transported as a vector to the nucleus of a new receptor cell and integrate them to its genome. The so-called “genetic therapy” would emerge years later after observing that integrated sequences could be recognized as by the cell as its own and expressed as functional proteins. It contributed to the idea that certain genetic diseases could be treated by substitution or repair of damaged DNA and, particularly in infections, destroy vital genes of pathogenetic agents. The clinical use of genetic therapy in humans created high expectations among the scientific and medical community. Nevertheless, there was certain discouragement within the medical field when patients showed complications after treatment. It seemed that such complications were the result of the inserted genes' expression and also of the viral genomes used as vectors. Furthermore, the inadequate insertion of genetic sequences into the patient's genome control sites caused alterations at the molecular level with physiological consequences which resulted fatal in most cases.

The drawbacks encountered in the administration of genetic therapy encouraged scientists to search for new clinical treatments and biomedical science as a whole has also continued to explore new strategies of genetic manipulation. It is so that in recent years, research has been focused on trying to inhibit a damaged gene expression rather than replacing such gene. This means that the synthesis of a protein coded by a damaged gene is prevented from taking place.

After an extremely hard research process it was found out that eukaryotic cells infected with several types of viruses have a genetic regulation mechanism which controls the presence of potentially expressible viral RNA molecules. Such mechanism, found in plants and fungi, constituted a breakthrough finding since it implied a new mechanism of antiviral immunity, unknown at that time. In addition to plants and fungi, some components of this mechanism were found in cells of invertebrates such as flatworms, protozoa like the ciliate paramecium, the Dictyostelium discoideum amoeba and some trypanosoma. It is known that this type of organisms do not have the ability to develop an immediate response mediated by interferon, typical of viral infections in vertebrates. Maybe this is the reason why those viral pathogens infect invertebrate organisms in a relatively easy manner. Thus, the mandatory question is how invertebrates respond to this type of infection. The answer is given by the same molecular system used by plants and fungi: RNA interference (RNAi) mechanism. This mechanism is induced by a double-stranded RNA molecule probably originated by replicative mediators coming from infecting viruses. RNA interference has been proposed as an evolutionally preserved mechanism which keeps genomic integrity of eukaryotic organisms, controls gene expression and provides protection against exogenous viral infections.

Important progress in this field was made in 1998 when a group of embryologists from Washington's Carnegie Institute studied the interference produced in genetic expression through the introduction of antisense RNA in the cells of Caenorhabditis elegans [Fire et al., Nature 391:806-1 I(1998)]. Since 1990 and based upon studies in plants, it is known that the pairing between antisense DNA or RNA and mRNA blocks its translation. What was original in this study was the introduction of double-stranded RNA into cells (i.e. hybrid sense-antisense RNA). Contrary to what was expected and to their surprise the group proved that genetic silencing effect in double-stranded RNA was much more powerful than in single stranded RNA. Even more surprising was to ascertain that the process stoichiometry did not match the idea of a mere blockage of mRNA by hybridization. Only a few double-stranded molecules are sufficient to silence genetic expression despite the larger quantity of mRNA existing within the cell. As a consequence, researchers focused on an amplifying or catalytic mechanism of unknown origin. Even though genetic silencing in animals is different from that produced in plants and fungi, the processes and molecular actors are essentially the same. The usual physiological silencing pathway starts upon the expression of a pre-mRNAi (microRNA precursor), that is a simple chain which can fold back on itself and form a double-stranded hairpin known as mRNAi. Another possibility is the presence of double stranded RNA probably derived from viruses or transposons. In both cases, these double stranded RNA are spliced by an endonuclease named Dicer into double stranded short fragments (19 to 24 nucleotide-long) known as sRNAis (short interfering RNA). The characterization of Dicer [Bernstein et al., Nature 409:363-6 (2001)] allowed its classification as RNAsaIII as well as the identification of an active site domain able to bind double stranded RNA within its structure.

SRNAis can bind with a complex named RISC(RNA-induced silencing complex), by activating it and producing a helicase activity which unwinds the two strands and uses one of the RNA strands as a template to degrade complementary RNA molecules. The resulting ribonucleic complex binds to the target mRNA. When complementarity is not perfect, RISC remains attached to the messenger and translation is attenuated. On the other hand, when it is perfect, RISC acts as a ribonuclease (RNAse), cleaving the messenger and releasing itself to repeat the process. This explains why a small number of sRNAi molecules can destroy a much larger number of mRNA.

Until very recently it was thought that RISC was a multiproteic complex comprised by unknown components. Tests pointed at the Argonaute protein family, a well conserved family in all eukaryotes, which members vary in number among species. At that time, it was known that some mutations found in specific components of this family caused defects in specific processes as well and on quite interesting occasions. That is the case of the Drosophila Piwi protein, one of the most representative members of the Argonaute family, which plays a fundamental role in the creation of molecular signals formed in the origin of GSC (germ-line stem cells) [Lin y Spradling, Development 124:2463-76 (1997)].

This process, among others, revealed that alteration occurs in specific silencing processes. However, the actual role of Argonautes within the process was still to be determined. It was in September 2004 that the Science Journal published the three-dimensional structure of a Piwi's homologous [Joshua-Tor et al., Science 305:1434-7. (2004)]. The authors showed the structural homology between a common domain for the Argonautes, the Piwi domain, and a RNAse domain, the H ribonuclease. It was concluded that the members of the Argonaute family were precisely the RISC, at least in their main activities, and that probably other peptides may also be involved by playing different roles.

Physiological functions known to the RNAi system are quite varied. Some of them are the defense system against viruses and the control of transposons or anomalous genetic elements.

On the other hand the mRNAi has revealed itself as an important mechanism in the endogenous expression regulation, as proved in many cases among different organisms. It is known that in Drosophila, the silencing is related to proliferation regulation mechanisms, cellular death and fat metabolism [Hay et al., Curr. Biology 13:790-795 (2003)]. Other example will be the control of left/right neuronal asymmetry in nematodes [Johnston and Hobert., Nature 426:845-9 (2003)], modulation of hematopoietic lineage differentiation [Bartel et al., Science 303:83-6 (2004)], or control of development of leaves and flowers in plants [Moussian et al., EMBO J. 17:1799-809. (1998)]. Although interference may probably be involved in other processes still to be described, it is important to point out that there is a close relationship between the development processes and house keeping mechanisms as well as stem cell differentiation, the latter likely to be modulated in some way by Argonaute proteins, as in the case of Drosophila. Thus, Dicer 1 gene genosuppressed (knock-out) mice cannot process the mRNAi, they die at an early stage without stem cells. Therefore, the RNAi system reveals as being essential for keeping the embryonic cell population through mechanisms not fully described yet. [Bernstein at al. Nat. Genet. 35:215-217 (2003)].

The characterization of Dicer [Bernstein at al., Nature 409:363-6 (2001)] allowed its classification as RNAseIII and showed the presence of an active site domain inside its structure able to bind to double stranded RNA.

The RNAi constitutes a powerful tool which is currently being developed in the therapeutic field, mainly based on two facts: all cells contain the required machinery to carry out the RNA interference and genes are potential targets. This technology has certain advantages over other types of therapies under research, like a rational development as from the detection of the aimed therapeutic target, an extremely specific action and a reduced number of secondary effects.

Currently there is general knowledge about the RNAi mechanism in plants and the different mechanisms which mammals (and other organisms) have kept throughout evolution. In plants, the increase of RNA beyond a threshold level, as determined for example by a viral infection or a transposon, is considered as unusual activates a RNA polymerase, dependant on the RNA, which synthesizes an excess strand, i.e. creating a double stranded RNA. Subsequent processes are common to plant and animals. This double stranded RNA is also recognized as foreign and sliced into fragments consisting of 21 to 23 oligonucleotides (small interference double stranded RNA or RNAi) having two prominent nucleotides at one end of each strand. The energy necessary to process the RNA (hydrolysis) is provided by the ATP (which is presumably required to unwind the RNA double strand). The double stranded sRNAi are then recognized by specific proteins creating a RNA-protein complex for which ATP is also required. This complex “watches over” all RNA produced in the cell, and upon encountering one which sequence complements one of the strands of the RNAi, it catalyses the mRNA destruction. Clearly, the destruction of the mRNAs brings about the loss of the message thereby preventing the protein from being synthesized, which works as having stopped the codifying gene.

It is also known that androgens produce varied metabolic responses by means of their androgen receptor (AR). The AR is a 110 kD nuclear receptor activated by specific ligands. AR expression, which occurs within a wide range of tissues, may be activated either by testosterone or dehydrotestosterone. Both bind to the receptor by different affinities. A structural analysis of the AR reveals it as a member of a steroid-receptor super family, including the thyroid hormones, vitamin D and retinoid receptors.

The AR is highly expressed in different types of reproductive cells and plays an essential role in the development and stability of sexual functions. It also expresses itself in tissues not involved in reproduction by regulating a series of enzymes and proteins. In addition, it has been asserted that an abnormal regulation of the AR gene is a significant cause of several hormonal disorders.

Synthesis-blocking compounds or androgen action might have a favorable participation in processes where androgenic action is important: hyperplasia, prostate carcinoma, acne, boldness, virilizing syndromes in women, early puberty and loss of the libido in individuals with alterations in their sexual behavior.

The inventors herein have synthesized new double-stranded RNAi which specifically bind to the AR and present antiandrogenic activity.

### BRIEF DESCRIPTION OF THE INVENTION

This invention refers to a RNA double-stranded oligonucleotide with antiandrogenic activity, selected from the group IRA-1, IRA-2, IRA-3, IRA-4, IRA-5, IRA-6 and IRA-7. Oligonucleotides IRA-1, IRA-2, IRA-3, IRA-4, in particular, are inhibitors with a high antiandrogenic activity whereas oligonucleotides IRA-5, IRA-6, IRA-7, IRA-4 present a moderate the antiandrogenic activity.

In a preferred embodiment of this present invention said double stranded RNA oligonucleotide constitutes part of the formulation of either of a pharmaceutical or a cosmetic composition. Wherein said composition, another object of this invention, comprises said double stranded RNA oligonucleotide in a quantity that it so effective that when administered to an individual in need of such treatment it blocks the androgen receptor.

The desirable form of administration is topical although transdermal or oral routes are also included within the range of the invention.

Another purpose of this invention is the use of the double stranded RNA oligonucleotide of the invention, selected among the oligonucleotides IRA-1, IRA-2, IRA-3, IRA-4, IRA-5, IRA-6 and IRA-7, to treat androgen-metabolism related diseases and to produce a pharmaceutical composition to treat androgen-metabolism related diseases. This invention also includes the use of a double stranded RNA oligonucleotide, selected among oligonucleotides IRA-1, IRA-2, IRA-3, IRA-4, IRA-5, IRA-6 y IRA-7, to treat hair loss and to provide a beneficial effect on skin, and to produce a cosmetic composition for treat hair loss or to provide a beneficial effect on skin.

### BRIEF DESCRIPTION OF FIGURES

FIG. 1 nucleotide sequences of the double stranded RNA oligonucleotides of the invention.

FIG. 2. Description of the structure of oligonucleotides IRA-1, IRA-2, IRA-3, IRA-4, IRA-5, IRA-6 y IRA-7, object of this invention.

FIGS. 2A, 2B y 2C. Description of an assay using oligonucleotides IRA-1, IRA-2, IRA-3 and IRA-4, according to this invention. It is observed that the expression of the reporter gene drops when under control of an androgen-responsive promoter at levels below 10% of residual expression (inhibition above 90%)

FIG. 2A b)

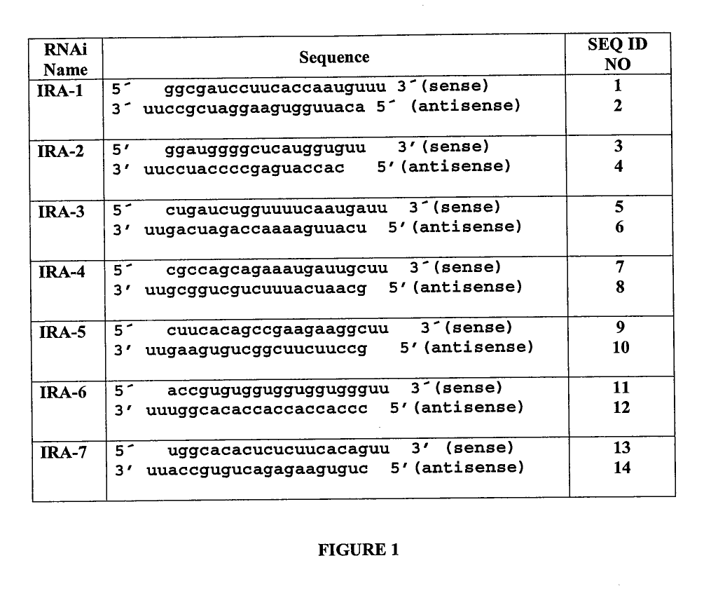

### AR Inhibition Expression by IRA-4. Biological Assay.

PC3AR cell line was transiently transfected with 0.05 μg of pARE-luc and 0.05 μg of pSport-Beta-gal according to LipofecAMINE 2000 protocol in 96-well plate. After 24 hs it was stimulated with mibolerone (mib) 10 nM. Fort-eight hours after transfection, luciferase and Beta-galactosidase (data standarization) activity were measured. Control is a siRNA with a sequence not homologous to that of the AR. Each step was made by triplicate where mean and standard deviations were represented.

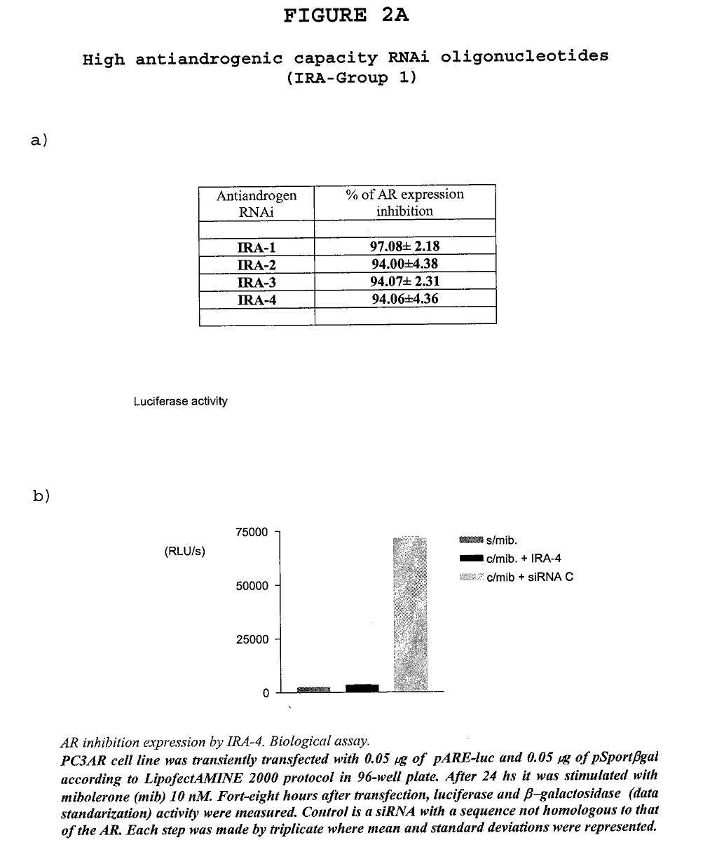

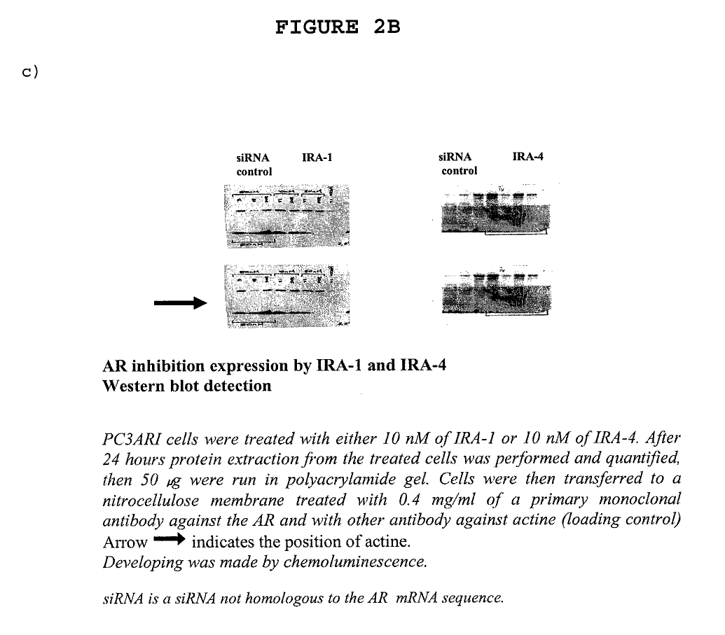

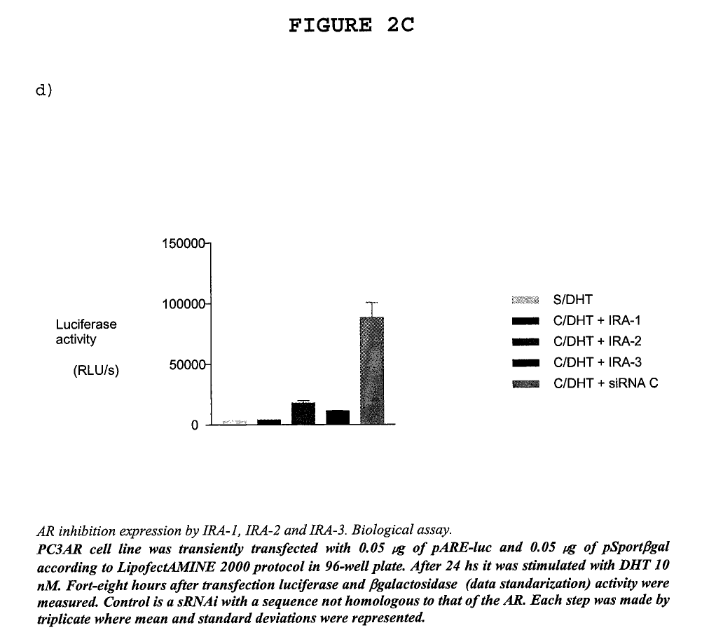

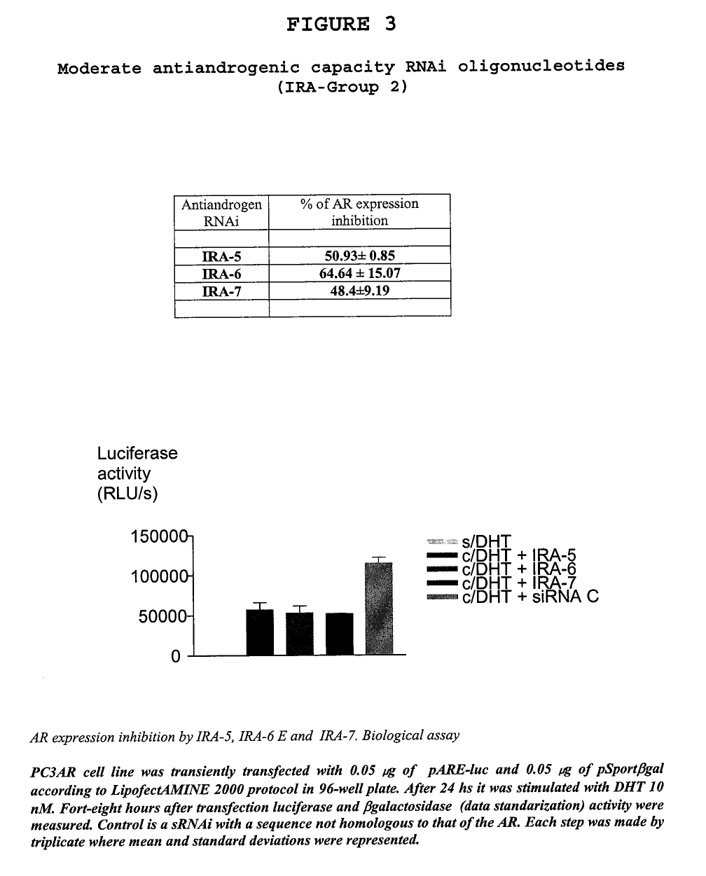

FIG. 2B c)

### AR Inhibition Expression by IRA-1 and IRA-4

### Western Blot Detection

PC3ARI cells were treated with either 10 nM of IRA-1 or 10 nM of IRA-4. After 24 hours protein extraction from the treated cells was performed and quantified, then 50 μg were run in polyacrylamide gel. Cells were then transferred to a nitrocellulose membrane treated with 0.4 mg/ml of a primary monoclonal antibody against the AR and with other antibody against actine (loading control) The arrow → indicates the position of actine.

Developing was made by chemoluminescence.

siRNA is a siRNA not homologous to the AR mRNA sequence.

FIG. 2C d)

### AR Inhibition Expression by IRA-1, IRA-2 and IRA-3. Biological Assay.

PC3AR cell line was transiently transfected with 0.05 μg of pARE-luc and 0.05 μg of pSport-Beta-gal according to LipofecAMINE 2000 protocol in 96-well plate. After 24 hs it was stimulated with DHT 10 nM. Fort-eight hours after transfection luciferase and Beta-galactosidase (data standarization) activity were measured. Control is a sRNAi with a sequence not homologous to that of the AR. Each step was made by triplicate where mean and standard deviations were represented.

FIG. 3. Description of an assay using oligonucleotides IRA-5, IRA-6, and IRA-7, according to this invention. It is observed that the expression of the reporter gene drops when under control of an androgen-responsive promoter at levels below 50% of residual expression (inhibition above 50%).

Moderate antiandrogenic capacity RNAi oligonucleotides (IRA-Group 2).

### AR Expression Inhibition by IRA-5, IRA-6 E and IRA-7. Biological Assay

PC3AR cell line was transiently transfected with 0.05 μg of pARE-luc and 0.05 μg of pSport [symbol] gal according to LipofecAMINE 2000 protocol in 96-well plate. After 24 hs it was stimulated with DHT 10 nM. Fort-eight hours after transfection luciferase and Beta-galactosidase (data standarization) activity were measured. Control is a sRNAi with a sequence not homologous to that of the AR. Each step was made by triplicate where mean and standard deviations were represented.

### DETAILED DESCRIPTION OF THE INVENTION

The invention refers to a double stranded oligonucleotide selected among the group consisting of oligonucleotides IRA-1, IRA-2, IRA-3, IRA-4, IRA-5, IRA-6 and IRA-7. They constitute ribonucleotides connected by phosphodiester bonds or alterations of such bonds. It also comprises chemical structures derived from nitrogenated bases and inherent riboses including inner alterations like 2′-O-alkyls, thiols, tandem alkyls all over the sequence or every even base. On the other hand, the chemical structure for the IRA-1, IRA-2, IRA-3, IRA-4, IRA-5, IRA-6 and IRA-7 double stranded RNA may not necessarily contain the protruding double uracil nucleotide at the 3′ sense and antisense strand, or any other nucleotide in such position, as thymidine or others.

Oligonucleotides IRA-1, IRA-2, IRA-3, IRA-4, IRA-5, IRA-6 and IRA-7 are described herein below:

IRA-1
5′ ggcgauccuucaccaauguuu 3′
3′ uuccgcuaggaagugguuaca 5′
IRA-2
5′ ggauggggcucaugguguu 3′
3′ uuccuaccccgaguaccac 5′
IRA-3
5′ cugaucugguuuucaaugauu 3′
3′ uugacuagaccaaaaguuacu 5′
IRA-4
5′ cgccagcagaaaugauugcuu 3′
3′ uugcggucgucuuuacuaacg 5′
IRA-5
5′ cuucacagccgaagaaggcuu 3′
3′ uugaagugucggcuucuuccg 5′
IRA-6
5′ accgugugguggugguggguu 3′
3′ uuuggcacaccaccaccaccc 5′
IRA-7
5′ uggcacacucucuucacaguu 3′
3′ uuaccgugucagagaaguguc 5′

This invention also includes the different variation, imitations and derivatives as well as those molecules sharing at least 10 oligonucleotides from the above described oligonucleotides IRA-1, IRA-2, IRA-3, IRA-4, IRA-5, IRA-6, IRA-7.

Surprisingly, the oligonucleotides from this invention have been detected to inhibit the androgen receptor (AR) expression according to certain capacity level. Oligonucleotides IRA-1, IRA-2, IRA-3, IRA-4 are particularly inhibitors presenting a high antiandrogenic activity, whereas in oligonucleotides IRA-5, IRA-6, IRA-7, the antiandrogenic activity is moderate. In terms of this invention, “high antiandrogenic activity” means such activity that allows the antiandrogenic expression to drop at least 80% and “moderate antiandrogenic activity” means such activity that allows the antiandrogenic expression to drop by 50%.

This distinguishing pharmacological ability is particularly useful in the treatment of androgen-related pathologies according to therapeutic needs. A high inhibiting capacity might be required in treatment of extreme severe pathologies, such as oncological diseases. Moderate antiandrogenic activity might, on the other hand, be desirable in dermo-cosmetic treatments.

The design of the oligonucleotides of this invention was based on the idea of aiming small RNAi molecules at specific regions of the androgen receptor (AR) mRNA's tertiary structure. Surprisingly, the molecules so designed, have shown to possess a certain degree of inhibition capacity on AR expression. As a result, it was possible to establish two separate groups of inhibitors which could turn out to be useful depending on the desired pharmacological grade to be obtained. The first group particularly presents a high inhibition capacity as it can be appreciated from the pharmacological assays on culture cell models.

In a preferred realization of this invention the double stranded RNA oligonucleotides of the invention could be formulated as components in a cosmetic or pharmaceutical composition in order to produce a beneficial effect upon skin or hair. Preferably, the composition includes at least one oligonucleotide in such amount that when administered to a subject in need of treatment it is effective so as to block the androgen receptor either totally or partially. This amount is preferred to range between 0.005% and 35% w/w or w/v. The composition may also include other usual active ingredients such as minoxidil in an amount to range between 1% and 20% w/w or w/v. The composition of the invention may also comprise at least one oligonucleotide combined with other therapeutically actives substances like finasteride and/or cosmetic substances which may contribute with a beneficial therapeutic or cosmetic effect.

The composition of the invention may be administered systemically; topical, oral or transdermal routes are preferred though.

Within the different embodiments of the invention, the topical or transdermal composition is preferably formulated as an aqueous or oil solution, as an emulsion, a gel, a cream, a lotion, a paste, an ointment, an aerosol, an occlusive patch, an emollient, etc. Depending on the pharmaceutical form, said composition may also include acceptable supports like oily and water-soluble components such as self-emulsifying wax, isopropyl myristate, cetyl alcohol, glycerin, methylparabene and propylparabene, and cosmetic and pharmaceutically acceptable skin-enhancing agents and antioxidants. In a preferred embodiment, the present invention comprises said double stranded RNA oligonucleotide in such quantity that it so effective that when administered to an individual in need of such treatment it blocks the androgen receptor.

Other skin-enhancing components of this topical or transdermal composition may take the form of emulsions, moisturizing forms, aminoacids, oligoelements and vitamins, all of which are widely known within the context of the invention and chosen by any person skilled in the technological area to which the invention pertains. If necessary, the formulation may also contain cosmetic and pharmaceutically acceptable antioxidants.

This said composition may also include other permeation or absortion promoters such as oleic acid, oleyl alcohol, ethyl alcohol, turpentines, terpenes, limonene, saponins, menthol and propylenglycol which are beneficial for improved skin absorption and are widely known within the context of the invention and chosen by any person skilled in the art. In addition, the composition of the invention may contain technically useful ingredients for improving absorption; examples are nanosomes, niosomes and cyclodextrins which are widely known within the context of the invention and chosen by any person skilled in the art.

As one of the alternatives of this invention, the composition may also be transported in colloidal, partitioned and biopolymer vectors, and more precisely in polyglycolic-polylactic acid, poly isobutyl cyanoacrylate, polyalginates or derivatives. Other possible carriers may be neutral, anyonic or catyonic, pH-sensitive or flexible liposomes. Furthermore, a preferred embodiment of the present invention comprises said double stranded RNA encapsulated into microsphere, nanosphere, oleosome, niosome, liposome or nanocapsule, nucleus or walls.

Included within the reach of this invention there is also a method for hair loss treatment consisting of a pharmaceutical or cosmetic composition which contains at least one of the oligonucleotides specified in this invention. The administration dose shall be determined by the attending physician depending on the excess of androgen manifested by the patient. As it is well known in the medical field, there are several factors affecting the dosage to be given, among them are: weight, age, gender, the oligonucleotide to be administered, time and route of administration, general health condition and concomitant medication.

The oligonucleotides included in this invention may be synthesized according to established procedures. In brief, synthesis can be performed by means of fluorescent labeling and the use of tert-butyldimethylsilyl (TBDMS) as protecting group. This procedure is similar to that of the DNA synthesis, except for the fact that an additional protecting group is required in the ribose 2′ hydroxyl position. This position may also be protected by using the chemical strategy of 2′-O-triisopropylsililoxymethyl (TOM) or 2-O-bis(2-acetoxyeti)methyl (ACE). This strategy is combined with other base protecting groups.

Next there follow some examples of the specifications contained within this invention and in vitro evidence regarding the antiandrogenic activity of the oligonucleotides herein specified.

### EXAMPLES OF SPECIFICATIONS

### Example 1

### Production of a PC3 Cell Line which Constitutively Expresses the cDNA of the Androgen Receptor (AR)

a) Sub-Cloning of cDNA from the Androgen Receptor (AR) into the pCIneo Vector.

The AR cDNA was inserted into the p5HbhAR vector. The AR cDNA containing 3100 by was subcloned into the expression vector pCIneo (Promega Corp., Madison, Wis., USA) which provides neomicin resistance (G-418). The p5HbhAR was digested with XbaI and partially digested with EcoRI in order to be sub-cloned into the PCIneo vector (pCIneoAR).

Restriction mapping and partial sequencing were performed so as to verify the correct insertion of cDNA into the PCIneo plasmid.

[symbol]

### b) PC3 Cell Culture

PC3 (ATCC® Number CRL-1435™) is a cell line originated from a primary culture of prostatic epithelial cells obtained from a subject showing a grade IV prostate adenocarcinoma [22363: Kaighn M E, et al. Establishment and characterization of a human prostatic carcinoma cell line (PC-3). Invest. Urol. 17: 16-23, 1979. PubMed: 447482]. This cell line was cultured with DMEN:F12 supplemented with BFS 10% and gentamicin [symbol] g/mL at 37° C. with 5% CO₂

### c) Transfection and Selection of a Stable Line

The PC3 cells were loaded into 10 cm² plates one day before transfection with a density high enough to obtain 50% cell confluency at the day of transfection. The media used was DMEM, supplemented with 10% fetal bovine serum and gentamicin [symbol] g/mL at 37° C. with 5% CO₂.

Two micrograms of pCIneoAR plasmid were incubated into a 48-well plate, using 100 [symbol] L of DMEM media. Five microliters of Lipofectin and 100 [symbol] L of DMEM media were added into another well and incubated during 45 minutes.

After incubation, both wells were combined and gently homogenated and then incubated again at room temperature for 15 minutes. After incubation 1.8 ml of DMEM media was added to the well containing the reaction Lipofectin-DNA. The mixture was gently mixed and the solution was poured into the plates containing the cells previously washed with serum-free and antibiotic-free DMEM.

The material was incubated for 6 hours in incubator. Once incubation was finished, the media was removed and DMEM: F12 with 10% fetal bovine serum (antibiotic-free) was added and kept for 48 hours. After this period the cells presented confluence. The cells were segregated with trypsin and transferred to other plates of the same size using dilutions 1:10 and 1:20 with DMEM media supplemented with 10% fetal bovine serum and 400 [symbol] g/mL G418 (selection media).

### d) Clone Selection

Approximately 10 days after transfection the resistant clones started to become visible. High vacuum grease was applied at the base of 5 mm plastic cylinders in order to isolate the clone area, subsequently remove the clone and transfer it to a separate well.

[symbol]

As the clones were isolated, they were transferred to a 12-well plate with selective media in 20% of fetal bovine serum.

Clones were kept under these conditions until they were dense enough to expand them in E-well plates. Media was kept with 400 [symbol] g/mL of G418 during approximately five passages (one month). The antibiotic amount was then reduced (200 [symbol] g/mL) for a similar period and was finally replaced by gentamicin 50 [symbol] g/mL.

### Example 2

### Detection of AR Protein by Western Blot

Around one million cells from each clone were loaded for study purposes into a 100 mm plate. They were incubated to sub confluence, washed with PBS 1× and finally were removed from the plate. They were centrifuged and resuspended in 70 [symbol] L of total protein extraction buffer (125 mM Tris-HCl pH 8.0, 2% SDS, 5% Triton X-100, 2 mM PMSF). Total protein concentration was determined by the Lowry method [Lowry et al., 1951].

Two hundred micrograms from these samples were loaded and separated by electrophoresis in a 8% SDS-Page gel (acrylamide-bisacrylamide, 29:1). The Rainbow™ molecular weight marker was used.

The samples were transferred to a nitrocellulose membrane (Hybond™), using transference buffer during 45 minutes at 300 mA. The transference was checked by red Ponceau staining and then incubated overnight with skimmed powdered milk 5% in PBS-Tween 20 as blocking solution, at 4° C.

AR detection was performed by means of a rabbit primary polyclonal antibody aiming the N-terminal region of the interest protein (rabbit polyclonal AR N-20 sc-816, Santa Cruz Biotechnology, Santa Cruz, Calif.). Five milliliters of a 1:500 antibody dilution were prepared in a PBS-Tween 20 solution. The membrane was incubated with the antibody, under constant gently shaking, during 2 hours at room temperature. Three consecutive 10 minute washings were performed using PBS-Tween 20 solution. The secondary rabbit anti-IgG antibody (Amersham) was added in a 1:1000 dilution (5 mL) in PBS-Tween solution and incubated for one hour. Two additional 10-minute washings were then made with PBS-Tween 20 and one washing was made with PBS 1×.

The membrane was developed by chemiluminescence (Hybond ECL™, Amersham). High sensitivity auto radiographic films were used (Hyperfilm ECL™, Amersham).

### Example 3

### Inhibition Assay by IRA Oligonucleotides Using Luciferase Reporter Gene

a) PC3ARII cells were loaded into 96-well plates at 80% confluence 6 hours before transfection. Cells were grown in DMEM:F12 supplemented with 10% FBS and gentamicin at 37° C. and 5% Co2.

Just before transfection, cells were washed with PBS and supplemented with fresh serum-free and antibiotic-free media. Then the cells were transiently transfected with 0.05 μg of pCMVSportBgal (Life Technologies, Inc. Gaithersburg, USA), 0.05 μg of pARELuc and 2.5 pmol of RNAi 2 μM (Ambion, Austin, USA) according to co-transfection SRNAi and plasmid DNA LipofectAMINE 2000 protocol (Invitrogen Corporation, Carlsbad, USA). After 24 h the cell media was replaced with 200 μl DMEM supplemented with 2.5% of FBS and 20 nM of DHT. Cells were stimulated with DHT for 24 h and then washed with PBS 1× and lysated according to the Luciferase Assay kit protocol (Promega, Madison, USA). A fraction of such lysate was used to measure luciferase activity whereas another fraction was used to measure B galactosidase activity (data standardization). Luciferase activity was measured by a Berthold FB 14 luminometer.

b) B galactosidase Assay

[symbol] galactosidase was measured by means of an ELISA reader at 420 nm after a 4-hour incubation. The reaction mixture was as follows:

10 μl of cell lysate
500 μl buffer PM2.
0.35 μl of β-mercaptoethanol
40 μl of water

### Buffer PM2:

### Na₂HPO₄.12H₂o 60 mM

### NaH₂PO₄ 23 mM

### KCl 10 mM

### MgSO₄.H₂O 1 mM

### Example 4

### Cosmetic or Pharmaceutical Formulations

The oligonucleotides IRA-1, IRA-2, IRA-3, IRA-4, IRA-5, IRA-6 and IRA-7 may be part of topical cosmetic formulations in association with retinol (Vitamin A) or its derivatives, as for example retinoic acid, so as to treat skin aging or sunlight-caused skin aging. They can also be associated with skin-moisturizing active ingredients such as medium or long chain fatty acids, and their derived amides: hydroxymethyl cellulose, hydroxyethyl urea or dimethicone. In cosmetic hair formulations, the oligonucleotides IRA-1, IRA-2, IRA-3, IRA-4, IRA-5, IRA-6 and IRA-7 may be associated with minoxidil or a copper active peptide. In pharmaceutical formulations indicated for acne treatment these oligonucleotides may be associated with anti-inflammatories such as betametasone or antibiotics like erythromycin or a combination of both.

### i) Facial Care Treatment Gel

Active ingredients: IRA 0.1%-0.5%, copper active peptide 10.05%.

Excipients and stabilizers: Water, glycerine 5%, carbomer 3%, benzyl alcohol 0.3%, sodium chloride 0.6%.

### ii) Facial and Body Emulsions

Active ingredients: IRA 0.1%, Squalane 5%, Dimethicone PEG 0.5%.

Excipients and stabilizers: water, cyclomethicone 6%, Propylene glycol 5%, (Methylparaben, Propylparaben, Butylparaben) 0.6%.

iii) Acne Fighting Cream

Active ingredients: IRA 0.5%, erythromicyn 3%, benzoil peroxide 5%.

Excipients and stabilizers: Water, Octyl Methoxycinnamate 5%, Propylene glycol 5%, cyclomethicone 5%, (phenoxyethanol, isopropylparaben, isobutylparaben y butylparaben) 1%, EDTA 0.1%

### iv) Hair Lotion

Active ingredients: IRA 0.1%-0.5%, Minoxidil 5%, Dimethicone 0.5%, Aloe vera herbal extracts 0.1%.

Excipients and stabilizers: Water, cetyl alcohol 0.3%, glycerine 5%, lanolin 2%, dicaprylyl maleate 0.5%, Hydroxyethylcellulose 5%, Benzalkonium chloride 0.01%, (methylparabene, propylparabene, ethylparabene) 0.8%, EDTA 0.1%.

1.-20. (canceled)

21. A cosmetic composition comprising at least one double stranded RNA oligonucleotide selected from the group consisting of IRA-1, IRA-2, IRA-3, IRA-4, IRA-5, IRA-6 and IRA-7, wherein said double stranded RNA oligonucleotide inhibits the expression of the androgen receptor, and supports.

22. The composition according to claim 21, comprising at least one double stranded RNA oligonucleotide selected from the group consisting of IRA-5, IRA-6 and IRA-7.

23. The composition according to claim 22, comprising at least one double stranded RNA oligonucleotide which inhibits the expression of the androgen receptor in an inhibition percentage equal or lower than 65%

24. The composition according to claim 21, wherein it is formulated in the form selected from the group consisting of gel, emulsion, cream, paste, ointment, aerosol, occlusive patches and lotion.

25. The composition according to claim 21, wherein said supports are selected from the group consisting of oily, aqueous, hydrosoluble, promoting absorption, absorption enhancers components and the mixtures thereof.

26. The composition according to claim 21, wherein said supports are selected from the group consisting of liposomes, particles, colloids and polymers.

27. The composition according to claim 21, wherein the double stranded RNA oligonucleotide binds the RNAm of the androgen receptor.

28. A method of treatment to decrease hair loss, comprising the administration of the necessary quantity of at least one double stranded RNA oligonucleotide selected from the group consisting of IRA-1, IRA-2, IRA-3, IRA-4, IRA-5, IRA-6 and IRA-7, wherein said double stranded RNA oligonucleotide inhibits the expression of the androgen receptor.

29. The method according to claim 28, which comprises the administration of the necessary quantity of at least one double stranded RNA oligonucleotide selected from the group consisting of IRA-5, IRA-6 and IRA-7, wherein said double stranded RNA oligonucleotide inhibits the expression of the androgen receptor.

30. The method according to claim 29, wherein at least one double stranded RNA oligonucleotide inhibits the expression of the androgen receptor at a percentage of inhibition equal or higher than 65%.

31. The method according to claim 28, wherein a quantity of double stranded RNA oligonucleotide between 0.005% and 35% p/p is administered.

32. The method according to claim 28, wherein it is administered topically.

33. A pharmaceutical composition comprising at least one double stranded RNA oligonucleotide selected from the group consisting of IRA-1, IRA-2, IRA-3, IRA-4, IRA-5, IRA-6 and IRA-7, wherein said double stranded RNA oligonucleotide inhibits the expression of the androgen receptor; and excipients.

34. The composition according to claim 33, wherein at least one double stranded RNA oligonucleotide is selected from the group consisting of IRA-1, IRA-2, IRA-3 and IRA-4.

35. The composition according to claim 34, wherein at least one double stranded RNA oligonucleotide inhibits the androgen receptor expression in an inhibition percentage equal or higher than 70%.

36. The composition according to claim 33, wherein it is formulated in the form selected from the group consisting of gel, emulsion, cream, paste, ointment, aerosol, patches, solution, solid and lotion.

37. The composition according to claim 33, wherein the double stranded RNA oligonucleotide binds the RNAm of the androgen receptor.

38. A method of treatment for individuals who suffer from androgen metabolism disorders, comprising the administration of the necessary quantity of at least one double stranded RNA oligonucleotide selected from the group consisting of IRA-1, IRA-2, IRA-3, IRA-4, IRA-5, IRA-6 and IRA-7, wherein said double stranded RNA oligonucleotide inhibits the expression of the androgen receptor.

39. The method according to claim 38, comprising the administration of the necessary quantity of at least one double stranded RNA oligonucleotide selected from the group consisting of IRA-1, IRA-2, IRA-3 and IRA-4 wherein said double stranded RNA oligonucleotide inhibits the expression of the androgen receptor.

40. The method according to claim 39, wherein at least one double stranded RNA oligonucleotide inhibits the expression of the androgen receptor in a percentage of inhibition equal or higher than 70%.

41. The method according to claim 38, wherein a quantity of double stranded RNA oligonucleotide between 0.005 and 35% p/p is applied.

42. The method according to claim 38, wherein the administration is selected from the group consisting of topic, oral, sublingual, transdermal and endovenous administration.


SEQUENCE LISTING (14 sequences)

SEQ ID NO: 1 - RNA, 21 nt, Artificial Sequence
  completely synthesized oligonucleotide with antiandrogenic activity sense strand
ggcgauccuucaccaauguuu

SEQ ID NO: 2 - RNA, 21 nt, Artificial Sequence
  completely synthesized oligonucleotide with antiandrogenic activity antisense strand
acauuggugaaggaucgccuu

SEQ ID NO: 3 - RNA, 19 nt, Artificial Sequence
  completely synthetsized oligonucleotide with antiandrogenic activity sense strand
ggauggggcucaugguguu

SEQ ID NO: 4 - RNA, 19 nt, Artificial Sequence
  completely synthesized oligonucleotide with antiandrogenic activity antisense strand
caccaugagccccauccuu

SEQ ID NO: 5 - RNA, 21 nt, Artificial Sequence
  completelyl synthesized oligonucleotide with antiandrogenic activity sense strand
cugaucugguuuucaaugauu

SEQ ID NO: 6 - RNA, 21 nt, Artificial Sequence
  completely synthesized oligonucleotide with antiandrogenic activity antisense strand
ucauugaaaaccagaucaguu

SEQ ID NO: 7 - RNA, 21 nt, Artificial Sequence
  completely synthesized oligonucleotide with antiandrogenic activity sense strand
cgccagcagaaaugauugcuu

SEQ ID NO: 8 - RNA, 21 nt, Artificial Sequence
  completely synthesized oligonucleotide with antiandrogenic activity antisense strand
gcaaucauuucugcuggcguu

SEQ ID NO: 9 - RNA, 21 nt, Artificial Sequence
  completely synthesized oligonucleotide with antiandrogenic activity sense strand
cuucacagccgaagaaggcuu

SEQ ID NO: 10 - RNA, 21 nt, Artificial Sequence
  completely synthesized oligonucleotide with antiandrogenic activity antisense strand
gccuucuucggcugugaaguu

SEQ ID NO: 11 - RNA, 21 nt, Artificial Sequence
  completely synthesized oligonucleotide with antiandrogenic activity sense strand
accgugugguggugguggguu

SEQ ID NO: 12 - RNA, 21 nt, Artificial Sequence
  completely synthesized oligonucleotide with antiandrogenic activity antisense strand
cccaccaccaccacacgguuu

SEQ ID NO: 13 - RNA, 21 nt, Artificial Sequence
  completely synthesized oligonucleotide with antiandrogenic activity sense strand
uggcacacucucuucacaguu

SEQ ID NO: 14 - RNA, 21 nt, Artificial Sequence
  completely synthesized oligonucleotide with antiandrogenic activity antisense strand
cugugaagagacugugccauu